(19) United States
(12) Patent Application Publication
(10) Pub. No.: US 2010/0317021 A1
(43) Pub. Date: Dec. 16, 2010

(54) FLUIDIC ANTIBODY-CONTAINING DEVICES AND METHODS

(75) Inventors: Patrick A. Mach, Shorewood, MN (US); G. Marco Bommarito, Stillwater, MN (US); Ryan Patrick Simmers, Cottage Grove, MN (US); Brinda B. Badri, Woodbury, MN (US); Joseph J. Stoffel, Hastings, MN (US); Vinod P. Menon, Woodbury, MN (US)

Correspondence Address: 3M INNOVATIVE PROPERTIES COMPANY, PO BOX 33427, ST. PAUL, MN, 55133-3427, US

(73) Assignees: Vinod P. MENON, Saint Paul, MN (US); 3M INNOVATIVE PROPERTIES COMPANY, Saint Paul, MN (US)

(21) Appl. No.: 12/515,745

(22) PCT Filed: Nov. 20, 2007
(86) PCT No.: PCT/US2007/085252

§ 371 Date: Feb. 12, 2010

Related U.S. Application Data
(60) Provisional application No. 60/867,073, filed on Nov. 22, 2006.

(51) Int. Cl. G01N 33/53; C12M 1/34

(52) U.S. Cl. 435/7.1; 435/287.2

(57) ABSTRACT

The invention relates to devices and methods for analyzing a sample (and preferably preparing a sample), which is particularly used in analysis, such as analysis of a sample for a bacterium of interest.

### RELATED APPLICATION

The present application claims the benefit of U.S. Provisional Patent Application Ser. No. 60/867,073, filed on Nov. 22, 2006, and which is incorporated herein by reference in its entirety.

### BACKGROUND

The emergence of bacteria having resistance to commonly used antibiotics is an increasing problem with serious implications for the treatment of infected individuals. Accordingly, it is of increasing importance to determine the presence of such bacteria at an early stage and in a relatively rapid manner to gain better control over such bacteria. This also applies to a variety of other microbes.

Microbial detection from complex samples often requires sample treatment or preparation to facilitate down-stream detection. Sources of detection interference often need to be removed or altered in ways to change or eliminate their properties that interfere with analyte detection.

Commonly, physical/chemical methods are used to separate interfering sample components. Use of microsample treatments and delivery methods are not often available commercially or are designed for other applications so the methods are often not easy to use and may be imprecise leading to inaccurate detection down stream. Thus, devices and methods are needed for sample preparation and analysis.

### SUMMARY

The present invention provides devices and methods for sample analysis and optionally sample preparation. The devices herein can be lateral-flow devices, vertical-flow devices, or a combination thereof. In certain embodiments, the sample flow path includes at least two portions (which can be defined by two or more sample passage portions), which are oriented in different directions. For example, one can be oriented transverse to the other.

In one embodiment, the present invention provides: a device comprising: a sample flow path; a zone including a sample capture component that includes a mixture of two or more antibodies in the sample capture zone, wherein the two or more antibodies have antigenic specificities for two or more distinct target analytes (preferably, for two or more distinct analytes characteristic of a specific bacterium); one or more reagents for sample preparation disposed in one or more distinct zones of the sample flow path ahead of the sample capture zone; and optionally, a particulate analyte-binding material disposed in a distinct zone of the sample flow path ahead of the sample capture zone and different from the one or more sample preparation reagents. Preferably, the analyte-binding material comprises particulate material and one or more antibodies (and more preferably, two or more antibodies) having antigenic specificities for one or more distinct target analytes (characteristic of the detection objective, preferably, for two or more distinct analytes characteristic of a specific bacterium); wherein the antibodies of the sample capture zone and zone comprising an analyte-binding material have the same or different specificity.

Preferably, for such embodiments, the sample capture zone is disposed in or on a flow-through membrane. In certain embodiments, the reagent(s), and the optional particulate analyte-binding material are disposed on or in a flow-through membrane. In certain embodiments, the one or more (preferably, two or more) antibodies in the sample capture zone are chemically bonded to a flow-through membrane.

In another embodiment, the present invention provides a device for sample preparation and analysis of a target analyte, the device comprising: a sample flow path; one or more reagents for sample preparation disposed in one or more distinct zones of the sample flow path; a zone including an analyte-binding material comprising particulate material and one or more antibodies specific for one or more distinct target analytes, wherein the zone is disposed in the sample flow path downstream from at least one of the sample preparation reagents; and a zone including a sample capture component that includes one or more antibodies specific for one or more distinct target analytes, wherein the antibodies of the sample capture zone and zone comprising an analyte-binding material have the same or different specificity. Preferably, the analyte-binding material comprises particulate material and two or more antibodies specific for two or more distinct target analytes; and the sample capture zone comprises two or more antibodies specific for two or more target analytes, wherein the antibodies of the sample capture zone and zone comprising an analyte-binding material have the same or different specificity.

Preferably, for such embodiments, the sample capture zone is disposed in or on a flow-through membrane. In certain embodiments, the reagent(s), and the optional particulate analyte-binding material are disposed on or in a flow-through membrane. In certain embodiments, the one or more (preferably, two or more) antibodies in the sample capture zone are chemically bonded to a flow-through membrane.

In another embodiment of the present invention, there is provided a device for detecting the presence or absence of an analyte, the device including: a body including a flow path, a flow-through membrane, and a sample capture zone defined within or on the flow-through membrane; wherein the sample capture zone comprises a sample capture component comprising a mixture of two or more antibodies chemically bonded to the flow-through membrane; wherein the two or more antibodies have antigenic specificities for two or more distinct target analytes (preferably, for two or more distinct analytes characteristic of a bacterium).

In certain embodiments described herein, the device may further include one or more reagents for sample preparation disposed in one or more distinct zones of the sample flow path within the body of the device, wherein the one or more zones comprising sample preparation reagents are disposed in the sample flow path upstream from the sample capture zone.

In certain embodiments, the device may further include analyte-binding material disposed in one or more distinct zones of the sample flow path within the body of the device, wherein the one or more zones are disposed in the sample flow path upstream from the sample capture zone, and further wherein the analyte-binding material comprises particulate material and one or more antibodies specific for the one or more distinct target analytes. Preferably, the analyte-binding material comprises two or more antibodies having antigenic specificities for two or more distinct target analytes (characteristic of the detection objective, preferably, two or more distinct analytes characteristic of a specific bacterium). Preferably, the antibodies of the sample capture zone and zones comprising an analyte-binding material have the same or different specificity.

In certain embodiments of the devices described herein, the flow path comprises a first flow passage portion and a second flow passage portion forming a first and second flow path portions, wherein the flow-through membrane divides the first and second flow passage portions.

In certain embodiments, the devices herein further include a pressure source to induce flow from the first flow path portion to the second flow path portion past the sample capture component. The pressure source can be, for example, one of a syringe, vacuum source, absorbent pad, or capillary pressure.

In another embodiment, the present invention provides a device for detecting the presence or absence of an analyte, wherein the device includes: a body including a flow path and a plurality of layers to form a multiple-layered structure, the flow path defined by a first flow passage portion and a second flow passage portion formed between a first layer and a second layer; and a sample capture component disposed between the first and second layers, wherein the sample capture component comprises one or more antibodies having antigenic specificities for one or more distinct target analytes (preferably, for one or more distinct analytes characteristic of a specific bacterium). In certain embodiments, the sample capture component comprises two or more antibodies having antigenic specificities for two or more distinct target analytes (characteristic of the detection objective, preferably, for two or more distinct analytes characteristic of a specific bacterium).

In certain embodiments of a multiple-layered structure, the device further includes one or more intermediate layers between the first layer and the second layer, wherein the intermediate layer includes a patterned portion that forms at least one of the first and second flow passage portions. In certain embodiments, the device further includes a flow-through membrane disposed in an opening of at least one of the intermediate layers of the multiple-layered (i.e., multi-layered) structure. In certain embodiments, the device further includes an absorbent layer or portion between an intermediate layer and an outer layer to induce flow across the flow-through membrane.

In certain embodiments of a multiple-layered structure, the device includes first and second outer layers, a spacer layer, and an intermediate layer, wherein the intermediate layer is disposed between the first and the second outer layers, and the spacer layer is disposed between the first outer layer and the intermediate layer and forms a first flow passage portion along the multiple-layered structure. In certain embodiments, the device further includes a flow-through membrane disposed in an opening of the intermediate layer. In certain embodiments, the device further includes an absorbent layer or portion between an intermediate layer and an outer layer to induce flow across the flow-through membrane.

In certain embodiments of the multiple-layered structure, the first outer layer includes a see-though portion to view the sample capture component. In certain embodiments of the multiple-layered structure, the first and second flow passage portions are oriented in different directions. In certain embodiments of the multiple-layered structure, the second flow passage portion is orientated generally traverse to the first flow passage portion.

In devices disclosed herein, there can be one or more chambers (i.e., reservoirs or wells) disposed within the first flow passage portion. Preferably, at least one of the one or more chambers includes a sample preparation reagent disposed therein. In certain embodiments, the sample preparation reagents include a lysing agent, a mucolytic agent, a surfactant, or a combination thereof To facilitate mixing of the sample preparation reagents with the sample, flow path (particularly, the first flow passage portion of the flow path) is tortuous.

In certain embodiments of the devices described herein, the sample capture zone comprises a patterned layer in a form of one or more symbols or text. The sample capture zone is defined by the presence of the sample capture component, which includes one or more antibodies. The antibodies can be monoclonal, polyclonal, or a mixture thereof Preferably, the antibodies comprise at least one monoclonal antibody, and more preferably, the antibodies comprise at least two monoclonal antibodies.

Methods of use of such devices are also included. In one embodiment, a method of analyzing a sample for the presence or absence of an analyte, the method comprising: providing a test sample suspected of containing one or more target analytes; providing a device as disclosed herein, wherein the device comprises a sample capture component in a sample capture zone and one or more sample preparation reagents; inducing flow of a test sample from a first flow path portion to a second flow path portion downstream of the sample capture component; providing conditions effective for reaction between the test sample and at least one of the sample preparation reagents in the first flow path portion; exposing the test sample to the sample capture component under conditions effective to bind an analyte and/or particulate analyte-binding material having analyte attached thereto to the sample capture component and produce a detectable signal; and evaluating the sample capture zone for the presence or absence of the detectable signal.

In another embodiment of the invention, a method is provided that involves: providing a test sample suspected of containing one or more target analytes; providing a device as disclosed herein, wherein the device comprises a sample capture component in a sample capture zone; inducing flow of a test sample from a first flow path portion to a second flow path portion downstream of the sample capture component; exposing the test sample to the sample capture component to bind an analyte and/or particulate analyte-binding material having analyte attached thereto to the sample capture component to induce a detectable signal of the sample capture component; and evaluating the sample capture zone for the presence or absence of the detectable signal.

### Definitions

The terms “analyte” and “antigen” are used interchangeably and refer to small molecules, pathogenic, and non-pathogenic organisms, toxins, membrane receptors and fragments, volatile organic compounds, enzymes and enzyme substrates, antibodies, antigens, proteins, peptides, nucleic acids, and peptide nucleic acids. In certain preferred embodiments, they refer to various molecules (e.g., protein A) or epitopes of molecules (e.g., different binding sites of protein A), or whole cells of a microorganism, that are characteristic of the microorganism (i.e., microbe) of interest. These include components of cell walls (e.g., cell-wall proteins such as protein A, and Clumping Factor, which is a cell wall-associated fibrinogen receptor that is found in S. aureus), external cell components (e.g., capsular polysaccharides and cell-wall carbohydrates), internal cell components (e.g., cytoplasmic membrane proteins), etc.

The term “mucus-containing sample” or “mucosal test sample” refers to samples that include, or are derived from, mucosal membranes and mucosal tissues, which are used interchangeably and refer to the surfaces of the nasal (including anterior nares, nasopharyngeal cavity, etc.), oral (e.g., mouth), outer ear, middle ear, vaginal cavities, and other similar tissues. Examples include mucosal membranes such as buccal, gingival, nasal, ocular, tracheal, bronchial, gastrointestinal, rectal, urethral, ureteral, vaginal, cervical, and uterine mucosal membranes.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, an analyte-binding material that comprises “an” antibody can be interpreted to mean that the analyte-binding material includes “one or more” antibodies that bind different analytes.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The disclosed subject matter will be further explained with reference to the attached figures, wherein like structure or system elements are referred to by like reference numerals throughout the several views.

FIG. 1 illustrates an embodiment of a sample capture layer or portion on a substrate.

FIG. 2 illustrates a sample capture component similar to FIG. 1 including a patterned sample capture layer or portion.

FIG. 3 is a schematic illustration of a device including a flow path and a sample capture component.

FIG. 4 is a schematic illustration of a device including multiple flow paths and sample capture components.

FIG. 5 is a schematic illustration of a device including a syringe or pressure source to induce flow along a flow path of the device.

FIG. 6 is a schematic illustration of a device including a vacuum source to induce flow along a flow path of the device.

FIGS. 7-8 schematically illustrate embodiments of a lateral-flow device including a flow-through membrane which forms a flow path including a sample capture component between a first flow path portion and a second flow path portion of the device.

FIG. 9 schematically illustrates an embodiment of a device including a sample capture component on a flow-through membrane (i.e., a porous membrane) of the device.

FIG. 10 schematically illustrates an embodiment of a device including a sample capture component on a flow-through membrane separating multiple flow path portions formed within a vial.

FIG. 11 schematically illustrates the sample capture component or portion of the embodiment illustrated in FIG. 10.

FIG. 12 schematically illustrates an embodiment of a device having a multiple-layered structure and including a sample capture component on a flow-through membrane.

FIG. 13 is an exploded view illustrating the multiple-layered construction (i.e., multi-layered structure) for a device of the type illustrated in FIG. 12.

### DETAILED DESCRIPTION OF ILLUSTRATIVE EMBODIMENTS

In certain embodiments, the present invention is directed towards devices and methods of analyzing a test sample, and optionally preparing the test sample for analysis. Although preferably, the test sample is a mucosal test sample of a microorganism (i.e., microbe) of interest, devices and methods of the present invention have broader applicability with respect to the test sample, target analytes, and the subsequent use of the test sample.

In particular, the devices and methods of use of the present invention can involve not only detecting the presence of an analyte (e.g., characteristic of a microorganism of interest), but preferably identifying such analyte, which can lead to identifying, for example, a microbe for which the analyte is characteristic. In certain embodiments, devices and methods of use herein can involve analyzing the sample by quantifying the analyte.

Devices and methods of the present invention include the use of antibodies that are specific for the target analyte. The devices include a sample capture zone defined by the presence of a sample capture component. The sample capture component includes one or more antibodies (preferably, a mixture of two or more antibodies) specific for the analyte(s) of interest (i.e., target analyte(s)).

Preferably, devices and methods of use of the present invention include the use of a first set of two or more antibodies having antigenic specificities for two or more distinct analytes characteristic of the detection objective (preferably, characteristic of a specific microorganism of interest such as a bacterium), the use of a second set of two or more antibodies having antigenic specificities for two or more distinct analytes characteristic of the detection objective (preferably, characteristic of a specific microorganism of interest such as a bacterium), wherein, for each of the analytes present, at least one of the antigenic specificities of antibodies in the second set is not functionally blocked from binding to its analyte by the first set of antibodies, having a different antigenic specificity, with analyte bound thereto. Such antibodies are preferably cooperative in their binding characteristics. That is, they are capable of simultaneously binding to distinct regions of the target analyte(s) or optimally are found to be of complementary binding whereby the binding of their distinct analytes by one antibody set is enhanced by the binding of one or more other antibody sets.

Targets of interest can include microorganisms, although pure proteins and enzymes may also be of interest. Microorganisms (i.e., microbes) of interest include prokaryotic and eukaryotic organisms, particularly Gram positive bacteria, Gram negative bacteria, fungi, protozoa, mycoplasma, yeast, viruses, and even lipid-enveloped viruses. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp. as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Enterobacter sakazakii, E. coli O157 and multiple drug resistant Gram negative rods (MDR).

Gram positive and Gram negative bacteria are of particular interest. Of even more interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein, transport protein, enzyme, etc., responsible for antibiotic resistance.

Devices and methods of use of the present invention could be used to analyze a sample for separate molecules (e.g., molecules like protein A and Clumping Factor for analysis of Staphylococcus aureus) or two different epitopes of the same molecule (e.g., a protein).

Such analytes include, for example, cell-wall proteins such as protein A and microbial surface components recognizing adhesive matrix molecules (MSCRAMMs) such as fibrinogen-binding proteins (e.g., clumping factors), fibronectin-binding proteins, collagen-binding proteins, heparin-related polysaccharides binding proteins, and the like. Protein A and clumping factors, such as fibrinogen-binding factors and clumping factors A, B, and Efb, are also particularly useful for Staphylococcus aureus. Other cell-wall components of interest include capsular polysaccharides and cell-wall carbohydrates (e.g., teichoic acid and lipoteichoic acid).

Alternatively, other representative antigenic components of a microorganism of interest include, for example an internal cell component, which may or may not be associated with a cell membrane. Internal cell components are particularly useful in analyzing antibiotic resistant microbes, such as MRSA, VRSA, VISA, VRE, and MDR. Internal cell components that can be characteristic of such microbes include membrane proteins. Examples of such membrane proteins include cytoplasmic membrane proteins, outer membrane proteins, and cell membrane proteins. Cytoplasmic membrane proteins, such as penicillin binding proteins (PBP) (e.g., PBP2′ or PBP2a) can be particularly characteristic of antibiotic resistant microbes. For example, the cytoplasmic membrane protein PBP2′ is characteristic of MRSA.

Microorganisms of interest can be analyzed in a test sample that may be derived from a wide variety of sources, such as a physiological fluid, e.g., blood, saliva, ocular lens fluid, synovial fluid, cerebral spinal fluid, pus, sweat, exudate, urine, lactation milk, or the like. Further, the test sample may be derived from a body site, e.g., wound, skin, nares, scalp, nails, etc.

Samples of particular interest include mucus-containing samples, such as nasal samples (from, e.g., anterior nares, nasopharyngeal cavity, nasal cavities, anterior nasal vestibule, etc.), as well as samples from the outer ear, middle ear, mouth, rectum, vagina, or other similar tissue. Examples of specific mucosal tissues include buccal, gingival, nasal, ocular, tracheal, bronchial, gastrointestinal, rectal, urethral, ureteral, vaginal, cervical, and uterine mucosal membranes.

Besides physiological fluids, other test samples may include other liquids as well as solid(s) dissolved in a liquid medium. Samples of interest may include process streams, water, soil, plants or other vegetation, air, surfaces (e.g., contaminated surfaces), and the like.

The art describes various sampling techniques for samples comprising microbes, such as S. aureus. Such sampling techniques are suitable for the methods of the present invention as well. For example, it is common to obtain a sample from wiping the nares of a subject. A particularly preferred sampling technique includes the subject's (e.g., patient's) anterior nares swabbed with a sterile swab or sampling device. For example, one swab is used to sample each subject, i.e., one swab for both nares. The sampling can be performed, for example, by inserting the swab dry or pre-moistened with an appropriate solution into the anterior tip of the subject's nares and rotating the swab for two complete revolutions along the nares' mucosal surface.

A wide variety of swabs or other sample collection devices are commercially available, for example, from Puritan Medical Products Co. LLC, Guilford, Me., under the trade designation PURE-WRAPS, or from Copan Diagnostics, Inc., Murrietta, Calif., under the trade designations microRheologics nylon flocked swab and ESwab Collection and Transport System. A sample collection means such as that disclosed, for example, in U.S. Pat. No. 5,879,635 (Nason) can also be used if desired. Swabs can be of a variety of materials including cotton, rayon, calcium alginate, Dacron, polyester, nylon, polyurethane, and the like. The sample collection device (e.g., swab) can then be processed using the methods of the invention to prepare the mucosal test sample.

The sample collection device (e.g., swab) can then be cultured directly, analyzed directly, or extracted with an appropriate solution. Such extraction (i.e., elution) solutions typically include water and can optionally include a buffer and at least one surfactant. An example of an elution buffer includes, for example, phosphate buffered saline (PBS) with TWEEN 20 or with PLURONIC L-64. Other extraction solutions function to maintain specimen stability during transport from sample collection site to sample analysis sites. Examples of these types of extraction solutions include Amies' and Stuart's transport media.

The test sample (e.g., liquid) may be subjected to treatment prior to further analysis. This includes concentration, precipitation, filtration, centrifugation, distillation, dialysis, dilution, heating, inactivation of natural components, sonication, addition of reagents, chemical treatment, etc.

That is, the test sample can be prepared using a wide variety of means well-known to those of skill in the art. For example, the sample could be disrupted to make available for analysis an analyte characteristic of the specific microorganism of interest using physical means (e.g., sonication, pressure, boiling or other heating means, vortexing with glass beads, etc.). Alternatively, the sample could be disrupted to make available for analysis an analyte characteristic of the specific microorganism of interest using various chemical reagents, which can include one or more components.

In certain the methods of the invention, the sample (preferably mucosal sample) is combined with an enzymatic-lysing agent. Exemplary enzymes for lysing (i.e., enzymatic lysing agents) include lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, DNase, and RNase. Various combinations of enzymes can be used if desired. A preferred enzyme is selected from the group consisting of lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, and combinations thereof. Lysostaphin is particularly useful in methods of detecting the presence of Staphylococcus aureus.

Other lysing agents include salts (e.g., chaotrophic salts), solubilizing agents (e.g., detergents), reducing agents (e.g., beta-mercaptoethanol (BME), dithiothreitol (DTT), dithioerythritol (DTE), cysteine, tris(2-carboxyethyl)phosphine hydrochloride (TCEP; Pierce Chemical Company, Rockford, Ill.), n-acetyl cysteine)), acids (e.g., HCl), and bases (e.g., NaOH). Such lysing agents may be more suitable for certain organisms than for others, for example, they can be more suitable for use with Gram negative bacteria than with Gram positive bacteria.

Various combinations of such lysing agents and methods can be used if desired.

Methods of lysing are further discussed in U.S. Patent Publication No. 2005/0153370 A1. Lysostaphin is particularly useful in methods of detecting the presence of Staphylococcus aureus.

Additionally, if desired, and the sample is a mucus-containing sample, it can be further treated, either before or after lysing, with at least one reagent that can include a mucolytic agent. Treatment of mucus-containing samples with mucolytic agents can reduce the interference resulting from the presence of mucus during the analysis.

Examples of mucolytic agents include enzymes (e.g., pepsin, DNases, RNases, glucosidases, galactosidases, glycosidases), salts (e.g., chaotrophic salts), solubilizing agents (e.g., surfactants, detergents), reducing agents (e.g., beta-mercapto ethanol (BME), dithiotreotol (DTT), dithioerythritol (DTE), cysteine, TCEP, n-acetyl cysteine), and acids (e.g., HCl). Various combinations of such mucolytic agents can be used if desired. One of skill in the art will understand that there can be overlap between lysing agents and mucolytic agents; although not all lysing agents will be mucolytic, for example.

In certain embodiments, the mucosal sample and an enzymatic-lysing agent are incubated for a time sufficient to allow lysis of cells and release of at least some antigenic components of the cells; subsequently, the sample and enzymatic-lysing agent are combined with a mucolytic agent that is distinct from the enzymatic-lysing agent.

In a preferred embodiment, if the sample is a mucus-containing sample, after lysing, the sample can be contacted with a first reagent under conditions sufficient for reaction between one or more components of the mucus-containing sample and the first reagent to form a composition. In such an embodiment, the first reagent can include one or more reducing agents, preferably acidified (e.g., having a pH of less than 3). Examples of such reducing agents include beta-mercapto ethanol (BME), dithiothreitol (DTT), dithioerythritol (DTE), cysteine, TCEP, and n-acetyl cysteine. A preferred reducing agent is n-acetyl cysteine, which is preferred because it is relatively stable and can be oxidized easily. Reducing agents can be acidified using a variety of acids, such as inorganic acids (e.g., HCl) or organic acids (e.g., lactic acid, citric acid). Alternatively, if used in sufficiently high concentrations, the pH of the reducing agent does not need to be adjusted with an acid. Also, alternatively, an acid alone (e.g., HCl) can be used as the mucolytic agent.

Typically, but optionally, after adding a reducing agent, the sample preparation involves inactivating the reducing agent in the composition. The terms “inactivate” or “inactivating” or “inactivation” refer to stopping the activity of a reagent or stopping a reaction, for example, which can occur by a wide variety of mechanisms, including, for example, blocking, diluting, inhibiting, denaturing, competing, etc.

Inactivating can be done, for example, by providing a competitive substrate (for example, bovine serum albumen for n-acetyl cysteine). Other examples of reagents that inactivate the reducing agent include neutralizing buffers. Representative ingredients for neutralizing buffers can include, for example, buffering agent(s) (e.g., phosphate), salt(s) (e.g., NaCl), protein stabilizer(s) (e.g., BSA, casein, serum) polymer(s), saccharides, and/or detergent(s) or surfactant(s) (e.g., one or more of the following agents listed by tradenames and commonly available sources: NINATE 411 (amine alkylbenzene sulfonate, available from Stepan Co., Northfield, Ill.), ZONYL FSN 100 (Telomer B monoether with polyethylene glycol, available from E.I. DuPont de Nemours Co.), Aerosol OT 100% (sodium dioctylsulfosuccinate, available from American Cyanamide Co.), GEROPON T-77 (sodium N-oleyl-N-methyltaurate, available from Rhodia Novacare), BIO-TERGE AS-40 (sodium olefin (C₁₄-C₁₆)sulfonate, available from Stepan Co.), STANDAPOL ES-1 (sodium polyoxyethylene(1)laurylsulfate, available from Cognis Corp., Ambler, Pa.), TETRONIC 1307 (ethylenediamine alkoxylate block copolymer, available from BASF Corp.), SURFYNOL 465, 485, and 104 PG-50 (all available from Air Products and Chemicals, Inc.), IGEPAL CA210 (octylphenol ethoxylate, available from Stepan Co.), TRITON X-45, X-100, and X-305 (octylphenoxypolyethoxy ethanols, all available from The Dow Chemical Co.), SILWET L-7600 (polydimethylsiloxane methylethoxylate, available from Momentive Performance Materials, Inc., Wilton, Conn.), RHODASURF ON-870 (polyethoxylated(2)oleyl alcohol, available from Rhodia Novacare), CREMOPHOR EL (polyethyoxylated castor oil, available from BASF Corp.), TWEEN 20 and TWEEN 80 (polyoxyethylene sorbitan monolaurate and monooleate, both available from Sigma-Aldrich Corp.), BRIJ 35 (polyoxyethylene(23)dodecyl ether, available from Sigma-Aldrich Corp.), CHEMAL LA-9 (polyoxyethylene(9)lauryl alcohol, available from PCC Chemax, Piedmont, S.C.), PLURONIC L64 (poly(oxyethylene-co-oxypropylene) block copolymer, available from BASF Corp.), SURFACTANT 10G (p-nonylphenoxypoly(glycidol), available from Arch Chemicals Inc., Norwalk, Conn.), SPAN 60 (sorbitan monostearate, available from Sigma-Aldrich Corp.), CREMOPHOR EL (a polyethoxylated castor oil, available from Sigma-Aldrich Corp.). If desired, the neutralizing buffer can also be used to adjust the pH of the sample.

In addition to, or alternative to, a reducing agent, the sample preparation of a mucus-containing sample can include the use of one or more surfactants or detergents (e.g., subsequently to or concurrently with, the combining of the sample and the enzymatic lysing agent with the mucolytic agent).

For example, a reagent that includes an acidified reducing agent can be combined with a sample and the resultant composition can be contacted with a surfactant. Alternatively, the reagent comprising the acidified reducing agent can also include a surfactant.

Suitable surfactants can be nonionic, anionic, cationic, or zwitterionic. Representative surfactants include sodium dodecyl sulfate (SDS) and sodium lauryl sulfate (SLS). Preferably, the surfactant is an anionic surfactant. More preferably, the surfactant is SDS and/or SLS.

Optionally, the sample preparation method can include subsequently inactivating the surfactant. This can be done, for example, by providing a competitive substrate.

Other examples of inactivating the surfactant include using reagent-neutralizing buffers, such as a buffer that is sufficient to adjust the pH of the mucolytic test sample and surfactant to a pH of at least 5. Preferably, the buffer is sufficient to adjust he pH of the mucolytic test sample to a pH of no greater than 8.

Furthermore, if one or more of the sample preparation reagents is acidic, the subsequent composition including the analyte of interest is preferably neutralized to a pH of 7 to 7.5 or near 7.2. This can be done, for example, by providing a buffer and/or a diluent of the type described above. If the diluent is used, the inactivating and neutralizing steps can occur substantially simultaneously (e.g., upon the addition of the same reagent).

Optionally, inactivating the surfactant can be done substantially simultaneously with inactivating the reducing agent and/or neutralizing the resultant composition. This can be done using, for example, buffers as described above.

Other sample types of particular interest include wound exudate, urine, and cultured blood. Wound exudate samples can be typically acquired using a swab or a similarly designed sample acquisition device to contact a wound that has been cleansed using a saline wash. The swab sample can be eluted in an extraction solution. Such extraction (i.e., elution) solutions typically include water and can optionally include a buffer and at least one surfactant. An example of an elution buffer includes, for example, phosphate buffered saline (PBS) with TWEEN 20 or with PLURONIC L-64. Other extraction solutions function to maintain specimen stability during transport from sample collection site to sample analysis sites. Examples of these types of extraction solutions include Amies' and Stuart's transport media.

The eluted exudate test sample may be filtered prior to testing in order to remove cells and other non-bacterial components (i.e. red and white blood cells, skin cells, macroscopic debris) with sizes greater than 1 μm. The sample may be ready at this point for the assay as described herein. Other means of preparing the eluted wound exudate test sample may include adding flocculating agents to promote the precipitation of interfering proteins, while maintaining the bacteria in suspension. Another sample treatment possibility includes the use of differential lysing agents that will lyse eukaryotic cells without affecting bacterial cells. Lysing with such an agent may allow filtration with membranes smaller than 1 μm in pore size to capture and isolate the bacterial cells while flushing to waste the lysed components. The bacterial cells captured on the filter could then be eluted off that filter using an elution buffer similar to the ones described for elution of the original sample from a swab.

Physical methods may also be useful in preparing a wound exudate sample. For example, centrifugation may be used to separate interfering sample components greater in size than microbes, while maintaining the target bacteria in the supernatant. These sample treatment methods are know to those skilled in the art.

Urine samples could be treated in a slightly different manner. First, the sample may be collected using a fluid handling system rather than a swab. As such it would not necessarily require elution as described for a swab sample. However, the subsequent sample treatments including: filtration, flocculation, differential lysing, and centrifugation, as described above would be useful in coarsely separating interfering sample components from the bacteria of interest.

Cultured blood samples could be treated in a manner analogous to urine samples. For example, centrifugation is a common method used to separate red and white blood cells from plasma which is the component of interest in detection of bacterial content.

In additional embodiments of the invention, the method can further include a step of combining the test sample with a labeled recognition element, such that the presence of an analyte (such as the microorganism or an antigenic component of the microorganism) can be detected, and preferably quantitatively analyzed. Representative labeled recognition elements can comprise reactant molecules for analyte binding (e.g., an analyte-binding material that includes a microorganism-recognizing reagent such as a bacteria-recognizing reagent). Such reactant molecules include antibodies, lectins, enzymes, and receptors and other binding pair technologies, as well as other reactant molecules that recognize metabolic by-products (e.g., pH changes, detectable enzyme production). For example, in one embodiment, the sample can be contacted with one or more antibodies. Such antibodies can be attached to particulate material, a membrane, or other solid support material. In some embodiments, one or more antibodies, such as an S. aureus antibody, are employed as a S. aureus reactant. S. aureus antibody” refers to an immunoglobulin having the capacity to specifically bind a given antigen inclusive of antigen binding fragments thereof

As mentioned above, the target analytes (i.e., analytes or components of interest) can be detected by a reactant molecule (e.g., an S. aureus reactant molecule or a bacteria-recognizing reagent for S. aureus). In some embodiments, one or more antibodies, such as an S. aureus antibody, are employed as an S. aureus reactant. “S. aureus antibody” refers to an immunoglobulin having the capacity to specifically bind a given antigen inclusive of antigen binding fragments thereof The term “antibody” is intended to include whole antibodies of a wide variety of isotypes (e.g., IgG, IgA, IgM, IgE, etc.), and fragments thereof from vertebrate, e.g., mammalian species which are also specifically reactive with foreign compounds, e.g., proteins.

The antibodies can be monoclonal, polyclonal, or combinations thereof Antibodies can be fragmented using conventional techniques and the fragments screened for utility in the same manner as whole antibodies. Thus, the term includes segments of proteolytically cleaved or recombinantly prepared portions of an antibody molecule that are capable of selectively reacting with a certain protein. Non-limiting examples of such proteolytic and/or recombinant fragments include Fab, F(ab′)₂, Fv, and single chain antibodies (scFv) containing a VL and/or VH domain joined by a peptide linker. The scFv's can be covalently or non-covalently linked to form antibodies having two or more binding sites. Antibodies can be labeled with a wide variety of detectable markers (i.e., detectable moieties) known to one skilled in the art. In some aspects, the antibody that binds to an analyte one wishes to measure (the primary antibody) is not labeled, but is instead detected indirectly by binding of a labeled secondary antibody or other reagent that specifically binds to the primary antibody.

In certain preferred embodiment, methods of the present invention utilize a set of two or more antibodies having antigenic specificities for two or more distinct antigenic components, or for distinct epitopes of a common antigenic component, characteristic of the microorganism, wherein, for each of the antigenic components (or epitopes of the antigenic component), at least one of the antigenic specificities of antibodies is not functionally blocked from binding to its analyte by the first set of antibodies having a different antigenic specificity, with analyte bound thereto. Such antibodies are preferably cooperative in their binding characteristics. That is, they are capable of simultaneously binding to distinct regions or epitopes of antigenic components the target analyte(s) or optimally are found to be of complementary binding whereby the binding of their distinct antigens by one antibody set is enhanced by the binding of one or more other antibodies.

In certain embodiments, devices and methods of use herein utilize at least one antibody that binds to an antigenic component of S. aureus that is released upon lysis consequent to the combination of, for example, a mucosal sample with an enzymatic lysing agent (e.g., lysostaphin).

Various S. aureus antibodies are known in the art. For example, S. aureus antibodies are commercially available from Sigma-Aldrich and Accurate Chemical. Further, S. aureus antibodies are described in U.S. Pat. No. 6,177,084, including the monoclonal antibody referred to as MAb 12-9 antibody, available from Inhibitex, Inc. (Alpharetta, Ga.). In certain preferred embodiments, an antibody is selected from those described herein (e.g., selected from the group consisting of MAb-76, MAb-107, affinity-purified RxClf40, affinity-purified GxClf40, MAb 12-9), fragments thereof, and combinations thereof Such antibodies are also disclosed in U.S. patent application Ser. No. 11/562,759, filed on Nov. 22, 2006, and PCT App. Ser. No. US 07/84,736, both entitled “ANTIBODY WITH PROTEIN A SELECTIVITY,” and in U.S. patent application Ser. No. 11/562,747, filed on Nov. 22, 2006, and PCT App. Ser. No. US 07/84,739, both entitled “ANTIBODY WITH PROTEIN A SELECTIVITY,” and in U.S. Pat. App. Ser. No. 60/867,089, filed on Nov. 22, 2006 and U.S. patent application Ser. No. ______ (Attorney Docket No. 62611US005), filed on even date herewith, both of which are entitled “SPECIFIC ANTIBODY SELECTION BY SELECTIVE ELUTION CONDITIONS.”

Preferred antibodies are monoclonal antibodies. Particularly preferred are monoclonal antibodies that bind to Protein A of Staphylococcus aureus (also referred to herein as “S. aureus” or “Staph A”).

More particularly, in one embodiment suitable monoclonal antibodies, and antigen binding fragments thereof, are those that demonstrate immunological binding characteristics of monoclonal antibody 76 as produced by hybridoma cell line 358A76.1. Murine monoclonal antibody 76 is a murine IgG2A, kappa antibody isolated from a mouse immunized with Protein A. In accordance with the Budapest Treaty, hybridoma 358A76.1, which produces monoclonal antibody 76, was deposited on Oct. 18, 2006 in the American Type Culture Collection (ATCC) Depository, 10801 University Boulevard, Manassas, Va. 20110-2209, and was given Patent Deposit Designation PTA-7938 (also referred to herein as accession number PTA-7938). The hybridoma 358A76.1 produces an antibody referred to herein as “Mab 76.” Mab 76 is also referred to herein as “Mab76,” “Mab-76,” “MAb-76,” “monoclonal 76,” “monoclonal antibody 76,” “76,” “M76,” or “M 76,” and all are used interchangeably herein to refer to immunoglobulin produced by hybridoma cell line 358A76.1 as deposited with the American Type Culture Collection (ATCC) on Oct. 18, 2006, and assigned Accession No. PTA-7938.

In another embodiment, suitable monoclonal antibodies, and antigen binding fragments thereof, are those that demonstrate immunological binding characteristics of monoclonal antibody 107 as produced by hybridoma cell line 358A107.2. Murine monoclonal antibody 107 is a murine IgG2A, kappa antibody isolated from a mouse immunized with Protein A. In accordance with the Budapest Treaty, hybridoma 358A107.2, which produces monoclonal antibody 107, was deposited on Oct. 18, 2006 in the American Type Culture Collection (ATCC) Depository, 10801 University Boulevard, Manassas, Va. 20110-2209, and was given Patent Deposit Designation PTA-7937 (also referred to herein as accession number PTA-7937). The hybridoma 358A107.2 produces an antibody referred to herein as “Mab 107.” Mab 107 is also referred to herein as “Mab107,” “Mab-107,” “MAb-107,” “monoclonal 107,” “monoclonal antibody 107,” “107,” “M107,” or “M 107,” and all are used interchangeably herein to refer to immunoglobulin produced by the hybridoma cell line as deposited with the American Type Culture Collection (ATCC) on Oct. 18, 2006, and given Accession No. PTA-7937.

Suitable monoclonal antibodies are also those that inhibit the binding of monoclonal antibody Mab-76 to Protein A of S. aureus. The present invention can utilize monoclonal antibodies that bind to the same epitope of Protein A of S. aureus that is recognized by monoclonal antibody Mab-76. Methods for determining if a monoclonal antibody inhibits the binding of monoclonal antibody Mab-76 to Protein A of S. aureus and determining if a monoclonal antibody binds to the same epitope of Protein A of S. aureus that is recognized by monoclonal antibody Mab-76 are well known to those skilled in the art of immunology.

Suitable monoclonal antibodies are also those that inhibit the binding of monoclonal antibody MAb-107 to Protein A of S. aureus. The present invention can utilize monoclonal antibodies that bind to the same epitope of Protein A of S. aureus that is recognized by monoclonal antibody MAb-107. Methods for determining if a monoclonal antibody inhibits the binding of monoclonal antibody MAb-107 to Protein A of S. aureus and determining if a monoclonal antibody binds to the same epitope of Protein A of S. aureus that is recognized by monoclonal antibody MAb-107 are well known to those skilled in the art of immunology.

Suitable monoclonal antibodies are those produced by progeny or derivatives of this hybridoma and monoclonal antibodies produced by equivalent or similar hybridomas.

Also included in the present invention include various antibody fragments, also referred to as antigen binding fragments, which include only a portion of an intact antibody, generally including an antigen binding site of the intact antibody and thus retaining the ability to bind antigen. Examples of antibody fragments include, for example, Fab, Fab′, Fd, Fd′, Fv, dAB, and F(ab′)₂ fragments produced by proteolytic digestion and/or reducing disulfide bridges and fragments produced from an Fab expression library. Such antibody fragments can be generated by techniques well known in the art.

Monoclonal antibodies useful in the present invention include, but are not limited to, humanized antibodies, chimeric antibodies, single chain antibodies, single-chain Fvs (scFv), disulfide-linked Fvs (sdFv), Fab fragments, F(ab′) fragments, F(ab′)₂ fragments, Fv fragments, diabodies, linear antibodies fragments produced by a Fab expression library, fragments including either a VL or VH domain, intracellularly-made antibodies (i.e., intrabodies), and antigen-binding antibody fragments thereof.

Monoclonal antibodies useful in the present invention may be of any isotype. The monoclonal antibodies useful in the present invention may be, for example, murine IgM, IgG1, IgG2a, IgG2b, IgG3, IgA, IgD, or IgE. The monoclonal antibodies useful in the present invention may be, for example, human IgM, IgG1, IgG2, IgG3, IgG4, IgA1, IgA2, IgD, or IgE. In some embodiments, the monoclonal antibody may be murine IgG2a, IgG1, or IgG3. With the present invention, a given heavy chain may be paired with a light chain of either the kappa or the lambda form.

Monoclonal antibodies useful in the present invention can be produced by an animal (including, but not limited to, human, mouse, rat, rabbit, hamster, goat, horse, chicken, or turkey), chemically synthesized, or recombinantly expressed. Monoclonal antibodies useful in the present invention can be purified by any method known in the art for purification of an immunoglobulin molecule, for example, by chromatography (e.g., ion exchange, affinity, and sizing column chromatography), centrifugation, differential solubility, or by any other standard technique for the purification of proteins.

Suitable antibodies also include a high avidity anti-Staphylococcus aureus clumping factor protein polyclonal antibody preparation that detects recombinant clumping factor (rClf40) protein of S. aureus at a concentration of preferably at least 1 picogram per milliliter (pg/mL), and more preferably up to 100 pg/mL. Suitable antibodies also include a high avidity anti-Staphylococcus aureus clumping factor protein polyclonal antibody preparation demonstrating at least a 4-fold increase in detection sensitivity in comparison to a Staphylococcus aureus clumping factor protein antiserum.

In certain embodiments, a high avidity anti-Staphylococcus aureus clumping factor protein polyclonal antibody preparation is useful, wherein the high avidity anti-S. aureus clumping factor protein polyclonal antibody preparation is prepared by a method that includes obtaining antiserum from an animal immunized with recombinant clumping factor (rClf40) protein of S. aureus; binding the antiserum to a S. aureus clumping factor (Clf40) protein affinity column; washing the column with a wash buffer having 0.5 M salt and a pH of 4; and eluting the high avidity anti-S. aureus clumping factor protein polyclonal antibody preparation from the column with an elution buffer with a pH of 2. Herein, the high avidity anti-Staphylococcus aureus clumping factor polyclonal antibody preparations from rabbits and goats are referred to as affinity-purified RxClf40 and affinity-purified GxClf40, respectively. In some embodiments, the high avidity anti-Staphylococcus aureus clumping factor protein polyclonal antibody preparation may be obtained by a method that further includes enriching the antiserum for the IgG class of antibodies prior to binding the antiserum to a S. aureus clumping factor (Clf40) protein affinity column. Such enrichment may eliminate non-immunoglobulin proteins from the preparation and/or enrich for the IgG class of antibodies within the sample.

As used herein, antiserum refers to the blood from an immunized host animal from which the clotting proteins and red blood cells (RBCs) have been removed. An antiserum to a target antigen may be obtained by immunizing any of a variety of host animals. Any of a wide variety of immunization protocols may be used.

Antibody avidity is a measure of the functional affinity of a preparation of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀).

Analyte-binding material useful for labeling purposes typically includes a solid support material. Solid support materials can include particulate materials, membranes, gels (e.g., agarose), or other solid support materials such as the surfaces of tubes or plates.

Exemplary solid supports can include materials such as nitrocellulose, polystyrene, polypropylene, nylon, gold sols, and/or late particles, and the like. For certain embodiments, particulate material and membranes are preferred. Preferably, for certain embodiments, the analyte-binding material is particulate material (e.g., polystyrene and/or latex beads having an average particle size of less than 1 micron, and preferably, approximately 0.3 micron).

Typically, within an antibody-containing device is a sample flow path, and within the sample flow path is a sample capture zone. The sample capture zone is formed on or in a porous material within the sample flow path. Such porous material is also referred to herein generally as an analyte-binding material. It is preferably in the form of a membrane (e.g., a multi-layered material). Such porous material (preferably membrane) allows for fluid flow through the material. This fluid flow can also result in mixing of sample and reagents if desired, within the antibody-containing device.

The analyte-binding material can include a solid support material (for certain embodiments, it is a particulate material) having one or more antibodies disposed on the solid support. In certain embodiments, each particle of the particulate material has at least two antibodies that bind different analytes disposed thereon. For example, in certain embodiments, the analyte-binding material includes a solid support material (preferably particulate material) having antibodies MAb-76 and affinity-purified RxClf40 disposed thereon (for example, in a ratio of 1:1, 2:1, 1:2, 3:1, or 1:3) (the recognition element), and a detectable marker.

Suitable methods of attaching antibodies to the solid support include, for example, chemical attachment methods, particularly covalent attachment methods, such as disclosed in U.S. Pat. App. Pub. No. 2003/0162236 using cyanoborohydride chemistry in skim milk. Other chemical (e.g., covalent) attachment methods are also known and can be used, as well as physical attachment methods (e.g., passive absorption or adsorption).

Antibodies can be chemically attached to a support material, preferably a particulate support material, through either covalent attachment or non-covalent attachment. Chemical attachment can involve the use of functionalized solid support materials, which are commercially available. For example, magnetic beads functionalized with various groups such as carboxyl, amine, and tosyl are commercially available from Invitrogen (Carlsbad, Calif.) and Ademtech (Pessac, France). Streptavidin-coated particles are also available from several sources such as Invitrogen (Carlsbad, Calif.), Ademtech (Pessac, France), and Miltenyi Biotec GmbH (Bergisch Gladbach, Germany).

Non-covalent attachment of an antibody to a solid support material includes attachment by ionic interaction or hydrogen bonding, for example. One example of a non-covalent attachment included in the present invention is the well-know biotin-avidin (or streptavidin) system. Avidin-biotin affinity-based technology has found wide applicability in numerous fields of biology and biotechnology. The affinity constant between avidin and biotin is remarkably high (the dissociation constant, Kd, is approximately 10⁻¹⁵ M, see, Green, Biochem. J., 89, 599 (1963)) and is not significantly lessened when biotin is coupled to a wide variety of biomolecules. Numerous chemistries have been identified for coupling biomolecules to biotin with minimal or negligible loss in the activity or other desired characteristics of the biomolecule. A review of the biotin-avidin technology can be found in “Applications of Avidin-Biotin Technology to Affinity-Based Separation,” Bayer et al., J. of Chromatography, pgs. 3-11 (1990).

Representative methods for covalent attaching an antibody to a support material include utilizing functional groups in the support materials (such as carboxyl, amine, hydroxyl, maleimide, hydrazide) activated by activation compounds (such as glutaraldehyde, carbodiimide, cyanogens bromide) to react with another reactive groups (such as hydroxyl, amino, amido, or sulfhydryl groups) in an antibody. This bond may be, for example, a disulfide bond, thioester bond, amide bond, thioether bond, and the like. Antibodies may also be directly attached to support material functionalized with groups (such as tosyl, chloromethyl) that can directly react with a functional group on the antibody (such as amine)

Antibodies may be covalently bonded to a particulate support material by a wide variety of the methods known in the art. For example, beads derivatized with carboxyl groups are commercially available. Antibodies can then be coupled to these beads through the formation of an amide linkage between a primary amine on the antibody and the carboxyl groups on the bead surface. The coupling reaction is mediated by activation via carbodiimide.

The analyte-binding material can include various detectable markers suitable for the detection system desired. For example, such detectable markers (i.e., reporter or detectable moieties or labels) can include a fluorescent label (e.g., such as that described in U.S. Pat. App. Pub. No. 2003/0162236, which discloses fluorescent dye intercalated latex particles). Other detectable markers can include luminescent labels, magnetic labels, chromogenic labels, Raman active labels, and the like.

In one embodiment of the invention, the various reagents as discussed herein (e.g., lysing agents, mucolytic agents, labeling agents) can be disposed in dry form in a container such as a fluidic device, in particular an antibody-containing device. Such reagents can be dried down using various techniques, such as vacuum drying, and equipment, such as a convection oven and lyophilization.

For drying down reagents, a drying diluent can be used, if desired. An exemplary drying diluent can include, for example, a phosphate buffer, a disaccharide (e.g., trehalose, sucrose), optionally a polysaccharide (e.g., glycerol) specific to conjugation, and a preservative (e.g., sodium azide). Glycerol (i.e., glycerin) is preferably not used if faster drying is desired and/or if convection oven drying is used. A phosphate buffer is preferably present in an amount of at least 5 millimolar (mM), and more preferably at least 10 mM. It is preferably present in an amount of no greater than 500 mM, and more preferably no greater than 50 mM. A disaccharide is preferably present in an amount of at least 0.1 weight percent (wt-%), and more preferably at least 0.5 wt-%. A disaccharide is preferably present in an amount of no greater than 5 wt-%, more preferably no greater than 2 wt-%, and even more preferably no greater than 1 wt-%. A polysaccharide, if used, is preferably present in an amount of at least 1 wt-%. A polysaccharide, if used, is preferably present in an amount of no greater than 20 wt-%, and more preferably no greater than 10 wt-%. A preservative is preferably present in an amount of at least 0.01 wt-%, and more preferably at least 0.2 wt-%. It is preferably present in an amount of no greater than 0.8 wt-%, more preferably no greater than 0.5 wt-%, and even more preferably no greater than 0.1 wt-%.

The use of devices having reagents therein (particularly, dried-down reagents therein in solid or semi-solid form) can provide greater efficiency, less sample contamination, less sample loss through transfer, better stability, and longer shelf life.

As a result of the devices and methods of pretreatment (e.g., use of reagents in a fluidic device, lysing the cells to form cell-wall fragments, treatment of mucus-containing samples to reduce the interference resulting from the presence of mucus) samples having relatively low concentrations of the species of interest can be evaluated. Thus, advantageously, methods of the invention have improved sensitivity. For example, for certain embodiments, the test sample may include a relatively low concentration of microbes, particularly Staphylococcus aureus. Such relatively low concentrations include, for example, less than 5×10⁴ colony forming units (“cfu”) per milliliter (cfu/mL) of microbe, less than 5×10³ cfu/mL, less than 1000 cfu/mL, and even as low as 500 cfu/mL. Microbes, such as S. aureus, can be detected at high levels as well, ranging up to as much as 5×10⁷ cfu/mL, for example.

Depending on the techniques of analyzing used in the methods of the present invention, relatively small volumes of test sample can be used. Although test sample volume as high as 2 milliliters (mL) may be utilized, advantageously test samples on the order of 10 microliters (μL) are sufficient for certain methods, with 50-100 μL being preferred for certain embodiments.

Depending on the techniques of analyzing used in the methods of the present invention, the detection time can be relatively short. For example, the detection time can be less than 300 minutes, less than 250 minutes, less than 200 minutes, less than 150 minutes, less than 100 minutes, less than 60 minutes, and even as short as 10 minutes.

### Fluidic Device Designs

The following discussion of exemplary embodiments includes a sample capture component that includes one or more antibodies (preferably two or more antibodies having antigenic specificities for two or more distinct analytes characteristic of a specific bacterium) disposed in a device in a sample flow path. Optionally, other reagents used in detection (e.g., analyte-binding material including particulate material and one or more antibodies attached thereto) and/or reagents used in sample preparation (e.g., lysing agents, mucolytic agents, surfactants, or combinations thereof) can be disposed in the device in the sample flow path. Such reagents can be in solid or semi-solid form.

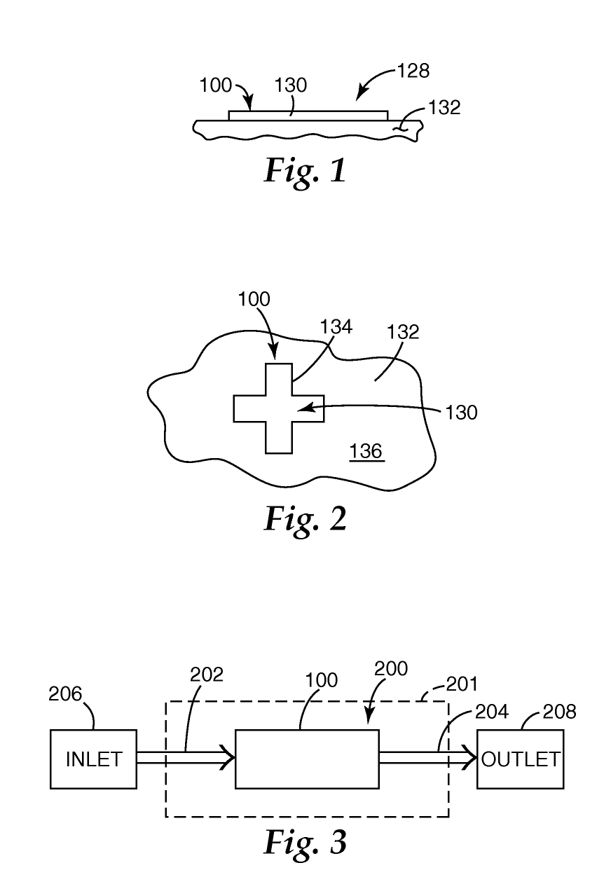

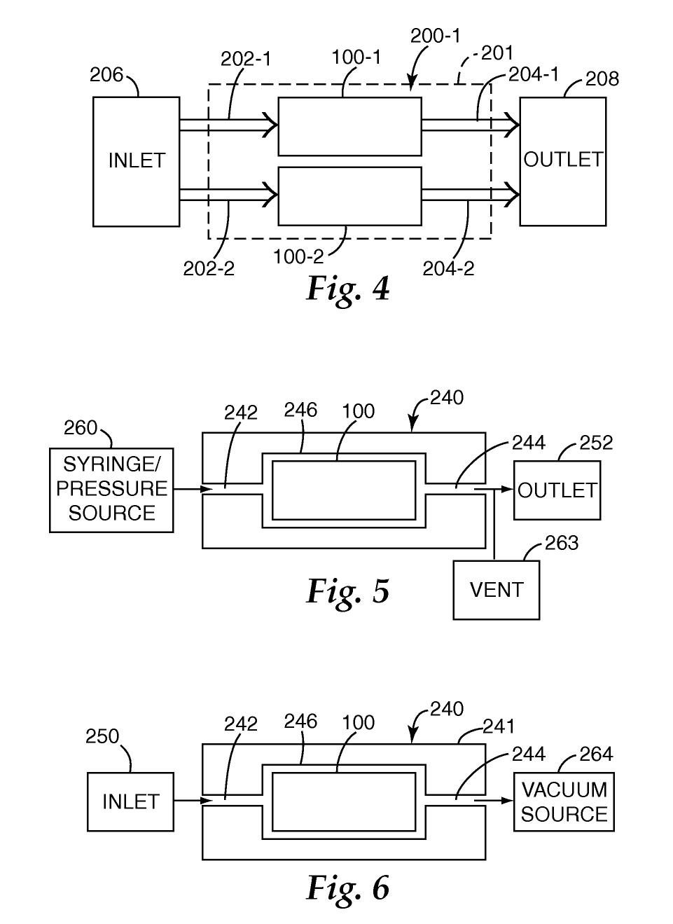

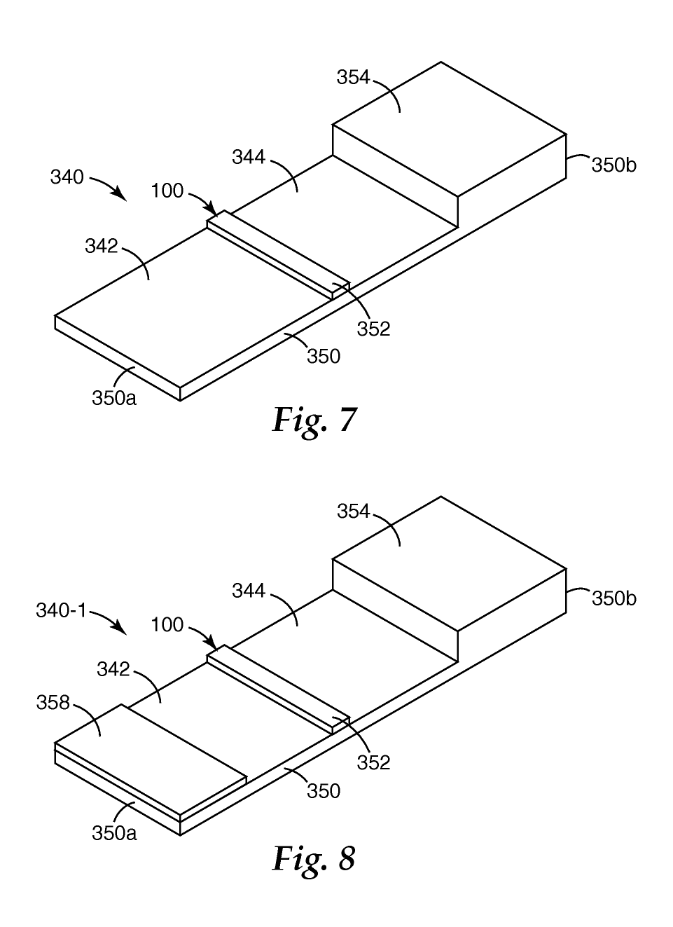

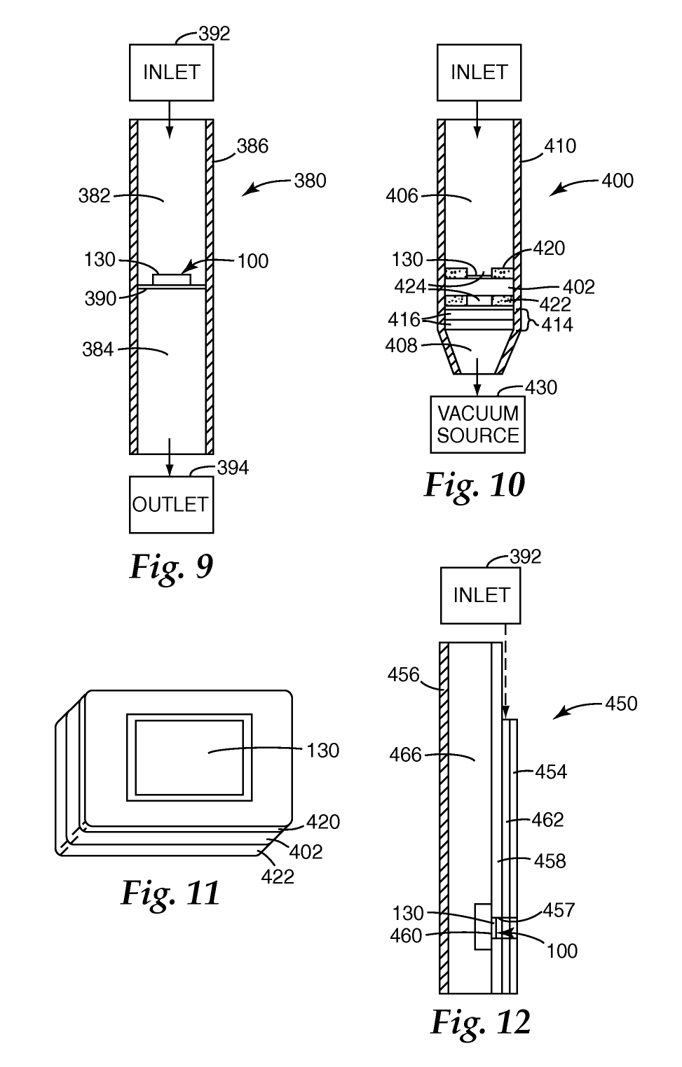

FIGS. 1-7 and 9 are generalized structures for greater understanding of the general concept of the sample capture component being in a flow path of a fluidic device: in solid form (FIG. 1); in the form of a design (FIG. 2); in one or more flow passages that create one or more flow paths (FIGS. 3-4); with optional flow generators (e.g., syringe/pressure/vacuum sources) shown (FIGS. 5-6); in a lateral-flow format (FIG. 7); or in a gravity-fed system (FIG. 9).

FIGS. 8 and 10-13 are more detailed structures for greater understanding of actual devices, how they are made, and how they would be used in methods described herein.

FIG. 1 illustrates a generalized representation of an exemplary embodiment wherein the sample capture component 100 is formed in a layer or portion 130 on a substrate 132, such as a thin film membrane, porous membrane (i.e., flow-through membrane), or other substrate. In one example, the sample capture component layer or portion 130 preferably includes one or more antibodies (preferably two or more antibodies) disposed on a thin film membrane or other substrate (preferably in or on a flow-through membrane formed from a porous material, for example).

In the embodiment illustrated in FIG. 2, the sample capture component 100 is deposited on the substrate 132 in a particular pattern to form one or more symbols or alphanumeric text 134. For example, in the illustrated embodiment, the sample capture component 100 is formed in the pattern of a “+” symbol 134 to indicate a positive test result. Upon binding with the analyte or particulate analyte-binding material, the “+” symbol 134 becomes visible relative to a background portion 136 to indicate a positive test result. The pattern of the symbol or text 134 is formed via known masking techniques to produce the deposited sample capture component layer or portion in the desired pattern and background portion 136 without the sample capture component layer or portion 130. Although FIG. 2 illustrates a “+” sign, application is not limited to any particular symbol or text.

The devices described herein utilize the sample capture component 100 as previously described to detect the presence of an agent in a test sample using, for example, a direct assay or indirect assay. In certain embodiments, in addition to the sample capture component (one or more antibodies) being disposed in a sample capture zone in the devices described herein, analyte-binding material (e.g., one or more antibodies attached to particulate material) may be disposed within the devices in one or more zones upstream of the sample capture component. Furthermore, one or more sample preparation reagents may be disposed within the devices in one or more zones upstream of the sample capture component (and typically upstream of the analyte-binding material). The materials in such zones (sample capture component, analyte-binding material, sample preparation reagents) are preferably disposed in or on a flow-through membrane.

FIG. 3 schematically illustrates one embodiment wherein a detection device 200 of the present application including a sample capture component 100 on a body 201 of the device 200. In the device 200 shown, the sample capture component 100 is disposed in a flow path (between a first flow path portion 202 and a second flow path portion 204) of the device 200. During use, a test sample flows along the first flow path portion 202 past the sample capture component 100 and then along the second flow path portion 204. As the test sample flows past the sample capture component 100, the analyte or particulate analyte-binding material having analyte attached thereto, for example, binds with the one or more antibodies contained within the sample capture component 100 to produce the detectable signal (e.g., due to the presence of a detectable marker such as a chromogenic or fluorimetric label, for example). As shown, the sample is injected into the flow path at inlet 206 and is collected or discharged from the second flow path portion 204 at outlet 208.

Although not shown, particulate analyte-binding material may be disposed in the sample flow path within the device upstream of the sample capture component (i.e., in the first flow path portion 202). Additionally, one or more sample preparation reagents (e.g., lysing agent, mucolytic agent) may be disposed in the sample flow path within the device upstream of the sample capture component (i.e., in the first flow path portion 202). The sample flow path portions, particularly the upstream or first flow path portion 202 can be tortuous, thereby facilitating mixing of the sample with any sample preparation reagents used (whether they are disposed in the device or not).

As shown in FIG. 3, the flow path for the test sample includes both a flow path portion upstream and downstream of the sample capture component 100 to induce flow past the sample capture component 100 for contact between the analyte(s) in the test sample and the sample capture component 100 to produce a detectable signal. The downstream portion is typically the waste stream. The reaction time and interaction between the analyte(s), any sample preparation reagents (if disposed in the device), a particulate analyte-binding material (if used and disposed in the device), and the sample capture component 100 is controlled based upon the flow rate of the sample past the sample capture component 100 and other variables discussed herein.

FIG. 4 schematically illustrates the device illustrated in FIG. 3, which includes multiple sample capture components 100-1, 100-2 on the same device 200-1 to detect the same or different analyte using a single device. For example, in a direct assay, the antibodies contained within sample capture components 100-1 and 100-2 can bind with different analytes in the test sample to detect the presence of different analyte(s) (characteristic of different organisms or substances) in the test sample or can bind to the same analyte(s). As shown in FIG. 4, sample capture components 100-1, 100-2 are also interposed in flow paths between the first flow path portions 202-1, 202-2 and the second flow path portions 204-1, 204-2, respectively. The test sample is introduced into the first flow path portions 202-1, 202-2 via inlet 206 and is discharged from the second flow path portions 204-1, 204-2 at outlet 208. Although FIG. 4 illustrates a single inlet 206 and outlet 208, multiple inlets and outlets can be used if desired for the multiple flow paths.

FIGS. 5-6 illustrate embodiments of a detection device 240 where the flow path is formed by a flow passage, which extends through a body 241 of the device. As shown, the flow passage includes a first flow passage portion 242 and a second flow passage portion 244. As shown, the first flow passage portion 242 is upstream of chamber 246 and the second flow passage portion 244 is downstream of the chamber 246. Sample capture component 100 is disposed in the chamber 246 in the flow path between the first flow passage portion 242 and the second flow passage portion 244. The test sample is injected into the first flow passage portion 242 flows from the first flow passage portion 242 through chamber 246 past the sample capture component 100 in chamber 246 to the second flow passage portion 244. Flow of the test sample past the sample capture component 100 allows the analyte to bind with the one or more antibodies (preferably, a mixture of two or more antibodies in the sample capture zone, wherein the two or more antibodies have antigenic specificities for two or more distinct analytes characteristic of a specific bacterium) in the sample capture component to produce the detectable signal responsive to the presence of the analyte or particulate analyte-binding material having analyte bound thereto, as previously described.

In the illustrated devices, the sensitivity of the sample capture component 100 is influenced by various factors including, for example, coating weight, flow rate of the test sample, concentration of the analyte or particulate analyte-binding material having analyte bound thereto, binding rate of the analyte or particulate analyte-binding material having analyte bound thereto, the cross sectional area of the flow path or passage and the pressure drop across the sample capture component 100 or along the flow passage or path.

Useful flow rates range from 2.5 microliters per minute (μL/min) to 1000 μL/min, most preferred flow rates are in the range from 25 μL/min to 250 μL/min.

In each of the illustrated embodiments, a time or period of exposure of the test sample to the sample capture component 100 is limited based upon the flow rate of the test sample across the sample capture component 100. Once the fluid flows past the sample capture component 100 it is no longer exposed to the sample capture component layer or portion, thus limiting exposure of the test sample to the sample capture component 100 to provide a relatively stable test result which does not vary significantly following conclusion of the test.

Flow through the flow passage portions, or flow along the flow path portions, can be induced by gravity or via capillary pressure, for example. Capillary flow can be imparted via a porous media or polymeric foam or through capillary channels or passages. The size and area of the passages can be designed to provide desired flow across the sample capture component.

Alternatively, flow can be actively induced via a pressure device or other pressure source as illustrated in FIGS. 5-6. In the embodiment schematically shown in FIG. 5, a syringe 260 is used to inject the test sample into the first flow passage portion 242. The test sample is injected via syringe 260 under pressure to induce fluid flow along the flow path through the first flow passage portion 242, the chamber 246, and the second flow passage portion 244. As shown in FIG. 5, the device includes a vent 263 open to the second flow passage portion 244 to allow escape of entrapped air or bubbles. The vent 263 can be an opening in fluid communication with the second flow passage portion 244 with a permeable or semi-permeable covering or opening with no covering. Alternatively, other techniques or devices can be used to reduce entrapped air bubbles or gas including, for example, priming techniques or release valves. In another example, the device itself can be oriented during testing so that air bubbles are naturally displaced.

In another embodiment illustrated in FIG. 6, fluid flow can be induced via a vacuum source 264. Vacuum sources of particular interest in these devices include, but are not limited to, those that rely on a mechanical action to generate a vacuum. For example, spring loaded mechanisms activated by the user in the form of levers or buttons; compressed elastomeric bladders that are allowed to regain their uncompressed state through a user activated action (such as the removal of a pressure sensitive adhesive strip). As shown, the vacuum source 264 is coupled to the second flow passage portion 244 to induce fluid flow along the flow path or flow passage.

FIG. 7 illustrates an embodiment of a lateral-flow detection device 340 including a sample capture component 100 and flow path as previously described. In the embodiment shown, the sample capture component 100 is interposed in the flow path between a first flow path portion 342 and a second flow path portion 344. As shown, the flow path is formed along a membrane 350 between opposed ends 350a and 350b of the membrane 350. The membrane 350 is formed of an absorbent body, such as a membrane formed from nitrocellulose, nylon, polystyrene, polypropylene, or other appropriate materials, having a pore size that facilitates flow along (i.e., flow through) the membrane 350 to form the flow path and the first and second flow path portions 342, 344 of the device 340. The sample capture component 100 includes a sample capture component layer or portion 352 that is deposited on the membrane along an intermediate portion of the membrane 100. In the illustrated embodiment, an absorbent pad 354 is coupled to the membrane 350 downstream of the sample capture component 100 to induce fluid flow along the membrane 350 from the first flow path portion 342 past the sample capture component 100 to the second flow path portion 344 (such downstream flow is typically a waste stream). The absorbent pad 354 can be made of a material such as glass fiber, cellulose, etc.

In an exemplary embodiment, the membrane is formed of a nitrocellulose material, for example. In an exemplary embodiment, the sample capture component layer or portion 352 is coated on the membrane 350 in a thin stripe averaging 2-3 millimeters (mm) in width and a having coating weight of 4-100 microliters per centimeter squared (μL/cm²) depending upon the configuration of the device. For use in an assay, sample preparation reagents (e.g., mucolytic or lysis reagents) would be spotted upstream of the analyte capture zone, near where the specimen is added to the nitrocellulose material, for example.

In an exemplary embodiment illustrated in FIG. 8, where like numbers are used to refer to like parts in FIG. 7, the device 340-1 includes a pad 358 upstream of the sample capture component 100. The pad 358 can include a particulate analyte-binding material that is mixed with a test sample. Alternatively, or additionally, the pad 358 could include one or more sample preparation reagents. The pad 358 can be made of a material such as glass fiber, cellulose, etc.

One or more sample preparation reagents can be added together or separately, in separate zones in the fluid path (of the devices of FIGS. 7 and 8) such that several specimen treatments can occur sequentially in the fluid path. These zones can be constructed by placing different materials coated with different sample preparation reagents in the path or by coating the materials directly in the fluid path. These constructions would allow for sequential specimen treatments in flow paths where this is advantageous for downstream detection.

Using the lateral-flow device shown in FIG. 7, in one embodiment, an analyte-binding material and a sample that contains the target analyte can be first mixed in a tube, for example, a micro-centrifuge tube, under conditions effective for the analyte-binding material to capture the target analyate and form a sample mixture. After mixing is completed, a first end of the membrane 350 (i.e., 350a) can be inserted into the micro-centrifuge tube containing sample mixture or a specimen aliquot can be transferred to the first end of the membrane 350 (i.e., 350a). At this point, the mixture will typically start to flow along the membrane 350 via capillary action. When the solution reaches the sample capture component 100, target analyte or analyte-binding material bound to the target analyte will be captured by the antibodies in the sample capture component 100 and this sample capture zone will display a detectable signal (e.g., a fluorescent or chromogenic signal).

The lateral-flow device shown in FIG. 8 can eliminate the need to mix the analyte-binding material with the sample containing the target analyte prior to contact with the device if the pad 358 includes the analyte-binding material. The analyte-containing sample can simply be dropped onto the conjugate pad 358 (containing the analyte-binding material) using a pipette or a syringe. As the analyte sample wets the pad 358, the analyte-binding material reconstitutes into the solution (or gets dispersed in the fluid of the sample) and can mix with the target analyte. The rest of this assay is identical to that described above.

Use of the exemplary device of FIG. 8 also allows one to use a sample preparation reagent such as a lysing agent, for example. In such a case, the lysing agent could be incorporated into the pad 358. As the analyte sample wets the pad 358, the lysing reagent reconstitutes into the solution and can mix with the target analyte to lyse it and release the analyte of interest.

Exemplary devices suitable for the lateral-flow embodiments disclosed herein are described, for example, in U.S. Pat. No. 5,753,517 or U.S. Pat. No. 6,509,196, and Published U.S. Application Nos. 2003/0162236 and 2003/0199004, for example.

In one embodiment of a lateral-flow device with sample preparation reagents disposed therein, the device includes: a sample flow path; a sample capture zone (preferably on or in a flow-through membrane) including a sample capture component that includes a mixture of two or more antibodies having antigenic specificities for two or more distinct target analytes (preferably, two or more distinct analytes characteristic of a specific bacterium); one or more reagents for sample preparation (e.g., a lysing agent, a mucolytic agent, a surfactant, or a combination thereof) disposed in one or more distinct zones of the sample flow path ahead of the sample capture zone; and optionally, a particulate analyte-binding material disposed in a distinct zone of the sample flow path ahead of (i.e., upstream of) the sample capture zone and different from the sample preparation reagent (i.e., wherein the sample preparation reagent is not just the particulate analyte-binding material).

In one embodiment of a lateral-flow device with sample preparation reagents disposed therein, the device for sample preparation and analysis of a target analyte includes: a sample flow path; one or more reagents for sample preparation disposed in one or more distinct zones of the sample flow path; a zone including an analyte-binding material comprising particulate material and one or more antibodies specific for one or more distinct target analytes, wherein the zone is disposed in the sample flow path downstream from at least one of the sample preparation reagents (but upstream of the sample capture zone); and a sample capture zone comprising one or more antibodies specific for one or more distinct target analytes, wherein the antibodies of the sample capture zone and zone comprising an analyte-binding material have the same or different specificity.

Such devices can be used for both sample preparation and analysis. Methods of use of such devices are analogous to those disclosed in U.S. patent No. 5,753,517 or U.S. Pat. No. 6,509,196, and Published U.S. Application Nos. 2003/0162236 and 2003/0199004, for example.

FIG. 9 schematically illustrates another embodiment of a detection device 380 that includes a sample capture component 100 in a flow path between a first flow passage portion 382 (defining a first flow path portion) and second flow passage portion 384 (defining a second flow path portion) within a body 386 of the device. In the embodiment shown, the sample capture component 100 includes a sample capture component layer or portion 130 on a flow-through membrane 390. The membrane 390 and sample capture component layer or portion 130 are disposed in the flow path and separate the first flow passage portion 382 and the second flow passage portion 384. In the illustrated embodiment, the sample is introduced into the first flow passage portion 382 at inlet 392 (illustrated schematically) and flows through the flow-through membrane 390 from the first flow passage portion 382 to the second flow passage portion 384. Sample flow is discharged from the second flow passage portion 384 at outlet 394. As described, the sample capture component layer or portion 130 includes the antibodies specific for binding with the analyte or particulate analyte-binding material as the sample flows past the sample capture component layer or portion 130 and through the flow-through membrane 390. Upon binding, the sample capture component 100 undergoes a detectable signal to detect the presence of the analyte or particulate analyte-binding material.

The flow-through membrane 390 can be a porous membrane with a relatively small pore size (e.g., 200 micrometers (μm)). Exemplary flow-through membranes can be formed of polyethersulfone (available under the trade designation SUPOR from Pall Corporation, Ann Arbor Mich.—0.2, 0.45 μm); polysulfone (I.C.E. or Tuffryn from Pall Corporation, Ann Arbor Mich.—0.4 μm); cellulose ester (MF Millipore from Millipore Corporation, Billerica Mass.—0.4 μm); polycarbonate (G.E. Polycarbonate Membranes from G.E. Osmonics, Minnetonka, Minn.—0.2 μm, 0.4 μm), or other material that has desired flow-through characteristics.

FIG. 10 illustrates another embodiment of a detection device 400 that includes a flow-through membrane 402 having a sample capture component layer or portion 130 separating a first flow passage portion 406 and a second flow passage portion 408 of the flow path. In the illustrated embodiment, the flow-through membrane 402 is disposed in a tube 410 which forms a body of the device 400 and the first and second flow passage portions 406, 408 of the device 400. In the illustrated embodiment, the flow-through membrane 402 is supported in tube 410 on a support 414 disposed in the flow path between the first and second flow passage portions 406, 408.

As shown in the illustrated embodiment, the support 414 includes a plurality of filter layers 416, which abut a tapered portion of the tube 410. Application, however, is not limited to the particular support 414 including the plurality of filter layers 416 as shown. The flow-through membrane 402 abuts the support 414. As cooperatively shown in FIGS. 10-11, opposed surfaces of the flow-through membrane 402 include adhesive layers 420, 422. The adhesive layer 422 connects the flow-through membrane 402 to support 414. As shown, the adhesive layers 420, 422 have a void or open space which cooperatively forms a passageway 424 between the first and second flow passage portions 406, 408. The passageway 424 is narrower than the first and second flow passage portions 406, 408 in order define a specific area of flow, and to concentrate sample flow to the sample capture component layer or portion 130, which is formed on the flow-through membrane 402 in the passageway 424.

Thus, for fabrication, the sample capture component layer or portion 130 is deposited within an inner area of the flow-through membrane 402 and the adhesive layers 420, 422 are positioned about the outer circumference of the flow-through membrane 402 to form the passageway 424. In the illustrated embodiment, the sample capture component layer or portion is deposited on a single side of the flow-through membrane 402 while the adhesive layer or portions 420, 422 are disposed on both sides of the flow-through membrane 402. However, application is not limited to the specific embodiments shown. As shown, flow is induced through the detection device 400 along the flow path and through the passageway 424 via a vacuum source 430. However, application is not limited to a vacuum source 430 to induce fluid flow and other techniques can be used, as previously described.

The device of FIGS. 10-11 can be used in the following manner For example, a sample containing the target analyte is first mixed with an analyte-binding material under conditions effective for the analyte-binding material to capture the target analyte and form a sample mixture. After completing this step, the sample mixture is introduced into the device of FIGS. 10-11 and allowed to flow through the sample capture component 130 and the flow-through membrane 402 at a given flow rate. As the sample solution passes through the sample capture component 130, analyte bound to the analyte-binding material can bind with the antibodies of the sample capture component, thereby displaying a detectable signal (e.g., color) in the zone containing the sample capture component 130.

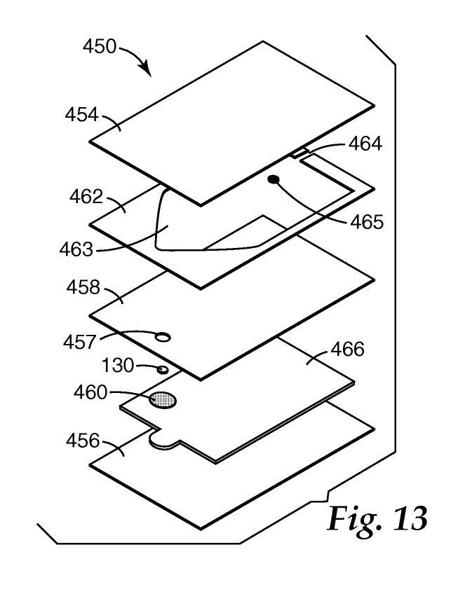

FIGS. 12-13 illustrate an embodiment of a detection device 450 (an enclosed vertical well device) having a sample capture component layer or portion 130 and flow-through membrane 460 where a body of the device is formed of a multiple layer construction. As shown, the multiple layer construction includes a face or first outer layer 454, a backing or second outer layer 456 and one or more intermediate layers. In the embodiment shown, the sample capture component 100 is supported proximate to an opening 457 through intermediate layer 458. Sample capture component layer or portion 130 is disposed on membrane 460, which is coupled to the intermediate layer 458 proximate to opening 457. The multiple-layered structure also includes a spacer layer 462 disposed between the face layer 454 and intermediate layer 458. The spacer layer 462 is patterned to form inlet 464 (shown in FIG. 13) and the first flow path portion. An absorbent layer 466 is disposed between the intermediate layer 458 and the backing layer 456 proximate to the opening 457 to induce fluid flow across a passageway formed through the flow-through membrane 460 in opening 457. In this embodiment, layers 454, 462, and 458 form an enclosed vertical well (i.e., reservoir or chamber) 463 with layers 454 and 458 forming the walls of the well 463 and the walls of the inlet 464.

As described, the first flow path portion is formed of a passage orientated along a length of the multiple-layered construction between the face layer 454 and the intermediate layer 458 to provide flow in a first direction. The device also includes a second flow path portion formed traverse to the first flow path portion to provide flow in a second direction generally transverse to the first direction across the flow-through membrane 460. In the illustrated embodiment, the face layer 454 can be formed of a transparent or see-through film so that the sample capture component 100 is visible to discern the detectable signal upon reaction of the analyte with the sample capture component 100. Alternatively, a portion of the face layer 454 can be transparent or see-through to view the sample capture component 100.

In the illustrated embodiment, fluid flow is induced across the flow-through membrane 460 via the absorbent layer 466. Layer 466 can be patterned to form an absorbent area downstream of the flow-through membrane 460 to form the traverse flow path or passage. Although FIGS. 12-13 illustrate a separate backing or outer layer 456, in alternate embodiments, the absorbent layer 466 can form the backing layer of the device, and application is not limited to the specific layers shown.

During use of the embodiments of FIGS. 12-13, a fluidic sample enters the enclosed vertical well 463 through inlet 464 and the fluid accumulates therein. If desired, sample preparation reagents (illustrated, for example, by the spot 465) can be placed at any locations in the fluid path that would allow for treatment prior to detection (i.e., in the fluid flow path upstream of the sample capture component 100). Although only one well (or reservoir) 463 is shown, this embodiment could include several different “reservoirs” that allow for fluid “accumulation.” This can facilitate sample preparation (i.e., treatment), for example, by either mixing with an analyte-binding material or sample preparation reagent in the reservoirs, either sequentially or simultaneously.

In each of the illustrated embodiments a time or period of exposure of the test sample to the sample capture component 100 is limited based upon the flow rate of the test sample across the sample capture component 100. Once the fluid flows past the sample capture component 100 it is no longer exposed to the sample capture component layer or portion, thus limiting exposure of the test sample to the sample capture component 100 to provide a relatively stable test result which does not vary significantly following conclusion of the test.

The device of FIGS. 12-13 can be constructed using the following materials: layer 456 can be a vinyl tape (SCOTCH Super 33 Plus Vinyl Electrical Tape available from 3M, St. Paul Minn.), layer 466 can be a glass fiber wicking material (Sterlitech GB 140 Glass Fiber, available from Sterlitech Corporation, Kent Wash.), layer 460 can be a 450-nm porosity polyethersulfone membrane (Pall SUPOR 450 Membrane, available from Pall Corporation, Ann Arbor Mich.), layer 458 can be a 0.8-mm thick polyvinyl chloride (PVC) backing material with a pressure sensitive adhesive on one side (Diagnostic Consulting Network Miba-010, available from Diagnostic Consulting Network, Irvine Calif.), layer 462 is a 1.6-mm thick 3M Polyethylene blown foam with a pressure sensitive adhesive on both sides (available from 3M Medical Division, 3M, St. Paul Minn.), and layer 454 can be a 3M Polyester General Use Transparency Film (available from 3M, St. Paul Minn.). To construct the detection device, each of the film layers can be die cut to its proper shape and size using a rotary die. The assembly begins by placing the flow-through filter membrane 460 over the opening 457 on the adhesive side of the intermediate layer 458. Next, the absorbent layer 466 can be placed over the filter membrane and positioned over the opening 457 on the adhesive side of the intermediate layer 458. This initial laminate can be placed absorbent layer 466 down on the adhesive side of the backing layer 456, applying pressure at the edges to ensure that the backing layer 456 adheres around the absorbent layer 466 to the intermediate layer 458, forming a seal. Next, the liner from one side of the spacer layer 462 can be removed and the adhesive side of the spacer layer 462 laminated to the non-adhesive side of the intermediate layer 458. Finally, the liner from the other side of the spacer layer 462 can be removed, and the outer layer 454 laminated to the adhesive layer on the spacer layer 462. A needle can be used to create two vent holes located at the top of the sample chamber.

The device of FIGS. 12-13 can be used in the following manner For example, a sample containing the target analyte is first mixed with an analyte-binding material under conditions effective for the analyte-binding material to capture the target analyte and form a sample mixture. After completing this step, the sample mixture is introduced into inlet port 464, accumulates in the well 463, changes direction of flow and passes through opening 457, the sample capture component 130, and the flow-through membrane 460 at a given flow rate. As the sample solution passes through the sample capture component 130, analyte bound to the analyte-binding material can bind with the antibodies of the sample capture component, thereby displaying a detectable signal (e.g., color) in the zone containing the sample capture component 130. In this embodiment, sample flow is typically assisted by the absorbent layer 466.

The discussion of exemplary embodiments herein above is primarily directed to a sample capture component disposed in a device in a sample flow path; however, other reagents used in detection (e.g., particulate analyte-binding materials) and/or reagents used in sample preparation (e.g., lysing agent, etc.) can be disposed in the device in the sample flow path as well. Reagents can be separated within such devices by a variety of well-known mechanisms. For example, a portion of a flow path can include one reagent (e.g., sample preparation reagent) and be separated from another portion of the flow path with another reagent therein (e.g., particulate analyte-binding material) by a valve therebetween made of a material (e.g., such as a hydrogel) that could dissolve upon contact with the sample. Other mechanisms of separation include membranes/materials of different porosities or fluid flow rates.

The embodiments described herein are exemplary in nature. It will be understood by one of skill in the art that other devices having other physical structures can be used to carry out the methods of the present invention. Furthermore, the specific devices described herein can be used in various methods (as would be appreciated by one of skill in the art) other than those specifically described.

The complete disclosures of all patents, patent applications, publications, and nucleic acid and protein database entries, including for example GenBank accession numbers, which are cited herein, are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

1. A device comprising:
a sample flow path;
a zone including a sample capture component comprising a mixture of two or more antibodies in the sample capture zone, wherein the two or more antibodies have antigenic specificities for two or more distinct target analytes;
one or more reagents for sample preparation disposed in one or more distinct zones of the sample flow path ahead of the sample capture zone; and
optionally, a particulate analyte-binding material disposed in a distinct zone of the sample flow path ahead of the sample capture zone and different from the one or more sample preparation reagents.

2. (canceled)

3. The device of claim 1, wherein the one or more reagents, and the optional particulate analyte-binding material are disposed on or in a flow-through membrane.

4. (canceled)

5. The device of claim 1, wherein the one or more reagents for sample preparation comprise a lysing agent, a mucolytic agent, a surfactant, or a combination thereof.

6.-7. (canceled)

8. The device of claim 1, which is a vertical-flow device.

9. The device of claim 1, wherein the sample flow path comprises at least two portions, one of which is transverse to the other.

10. A device for sample preparation and analysis of a target analyte, the device comprising:
a sample flow path;
one or more reagents for sample preparation disposed in one or more distinct zones of the sample flow path;
a zone including an analyte-binding material comprising particulate material and one or more antibodies specific for one or more distinct target analytes, wherein the zone is disposed in the sample flow path downstream from at least one of the sample preparation reagents; and
a zone including a sample capture component comprising one or more antibodies specific for one or more distinct target analytes, wherein the antibodies of the sample capture zone and zone comprising an analyte-binding material have the same or different specificity.

11. (canceled)

12. The device of claim 10, wherein the one or more reagents, and the optional particulate analyte-binding material are disposed on or in a flow-through membrane.

13. (canceled)

14. The device of claim 10, wherein the one or more reagents for sample preparation comprise a lysing agent, a mucolytic agent, a surfactant, or a combination thereof.

15. The device of claim 10, wherein the sample capture zone comprises a patterned layer in a form of one or more symbols or text.

16. (canceled)

17. The device of claim 10, which is a vertical-flow device.

18. The device of claim 10, wherein the sample flow path comprises at least two portions, one of which is transverse to the other.

19. The device of claim 10, wherein the analyte-binding material comprises particulate material and two or more antibodies specific for two or more distinct target analytes; and the sample capture zone comprises two or more antibodies specific for two or more target analytes, wherein the antibodies of the sample capture zone and zone comprising an analyte-binding material have the same or different specificity.

20. A device for detecting the presence or absence of an analyte, the device comprising:
a body including a flow path, a flow-through membrane, and a sample capture zone defined within or on the flow-through membrane;
wherein the sample capture zone comprises a sample capture component comprising a mixture of two or more antibodies chemically bonded to the flow-through membrane;
wherein the two or more antibodies have antigenic specificities for two or more distinct target analytes.

21.-22. (canceled)

23. The device of claim 20, further comprising one or more reagents for sample preparation disposed in one or more distinct zones of the sample flow path within the body of the device, wherein the one or more zones comprising sample preparation reagents are disposed in the sample flow path upstream from the sample capture zone.

24. The device of claim 20, further comprising analyte-binding material disposed in one or more distinct zones of the sample flow path within the body of the device, wherein the one or more zones are disposed in the sample flow path upstream from the sample capture zone, and further wherein the analyte-binding material comprises particulate material and one or more antibodies specific for one or more target analytes.

25. The device of claim 24, wherein the analyte-binding material comprises two or more antibodies specific for two or more distinct target analytes, wherein the two or more antibodies have antigenic specificities for two or more distinct target analytes.

26. The device of claim 24, wherein the antibodies of the sample capture zone and zones comprising an analyte-binding material have the same or different specificity.

27. (canceled)

28. The device of claim 20, wherein the flow path comprises a first flow passage portion and a second flow passage portion forming a first and second flow path portions, wherein the flow-through membrane divides the first and second flow passage portions.

29. The device of claim 28 further comprising a pressure source to induce flow from the first flow path portion to the second flow path portion past the sample capture component.

30.-31. (canceled)

32. The device of claim 20, which is a vertical-flow device.

33. The device of claim 20, wherein the sample flow path comprises at least two portions, one of which is transverse to the other.

34. The device of claim 20, wherein the one or more reagents for sample preparation comprise a lysing agent, a mucolytic agent, a surfactant, or a combination thereof.

35. A device for detecting the presence or absence of an analyte, the device comprising:
a body including a flow path and a plurality of layers forming a multiple-layered structure,
the flow path defined by a first flow passage portion and a second flow passage portion formed between a first layer and a second layer; and
a sample capture component disposed in a sample capture zone between the first and second layers, wherein the sample capture component comprises one or more antibodies have antigenic specificities for one or more distinct target analytes.

36. The device of claim 35 wherein the sample capture component comprises two or more antibodies have antigenic specificities for two or more distinct target analytes.

37. The device of claim 35 further including one or more intermediate layers between the first layer and the second layer, wherein the intermediate layer includes a patterned portion that forms at least one of the first and second flow passage portions.

38. The device of claim 37 further comprising a flow-through membrane disposed in an opening in at least one of the intermediate layers.

39. The device of claim 35, wherein the multiple-layered structure includes first and second outer layers, a spacer layer, and an intermediate layer, wherein the intermediate layer is disposed between the first and the second outer layers, and the spacer layer is disposed between the first outer layer and the intermediate layer and forms a first flow passage portion along the multiple-layered structure.

40. The device of claim 39 further comprising a flow-through membrane disposed in an opening of the intermediate layer.

41. The device of claim 38 further comprising an absorbent layer or portion between an intermediate layer and an outer layer to induce flow across the flow-through membrane.

42. (canceled)

43. The device of claim 35, further comprising one or more chambers disposed within the first flow passage portion.

44. The device of claim 43 wherein at least one of the one or more chambers includes a sample preparation reagent disposed therein.

45. The device of claim 35, wherein the first flow passage portion is tortuous.

46. The device of claim 35, wherein the first and second flow passage portions are orientated in different directions.

47. A method of preparing and analyzing a sample for the presence or absence of an analyte, the method comprising:
providing a test sample suspected of containing one or more target analytes;
providing a device of claim 1, wherein the device comprises a sample capture component in a sample capture zone and one or more sample preparation reagents;
inducing flow of a test sample from a first flow path portion to a second flow path portion downstream of the sample capture component;
providing conditions effective for reaction between the test sample and at least one of the sample preparation reagents in the first flow path portion;
exposing the test sample to the sample capture component under conditions effective to bind an analyte and/or particulate analyte-binding material having analyte attached thereto to the sample capture component and produce a detectable signal; and
evaluating the sample capture zone for the presence or absence of the detectable signal.

48. The method of claim 47, wherein the test sample comprises a mucus-containing sample.

49. A method of detecting the presence or absence of an analyte, the method comprising:
providing a test sample suspected of containing one or more target analytes;
providing a device of claim 20, wherein the device comprises a sample capture component in a sample capture zone;
inducing flow of a test sample from a first flow path portion to a second flow path portion downstream of the sample capture component;
exposing the test sample to the sample capture component under conditions effective to bind an analyte and/or particulate analyte-binding material having analyte attached thereto to the sample capture component and produce a detectable signal; and
evaluating the sample capture zone for the presence or absence of the detectable signal.

50. The method of claim 49, wherein the test sample comprises a mucus-containing sample.
